(19) United States
(12) Patent Application Publication
(10) Pub. No.: US 2024/0374262 A1
(43) Pub. Date: Nov. 14, 2024

(54) BIORESORBABLE DEVICE

(71) Applicant: ABIOMED, Inc., Danvers, MA (US)

(72) Inventor: Christopher KORKUCH, Danvers, MA (US)

(73) Assignee: ABIOMED, Inc., Danvers, MA (US)

(21) Appl. No.: 18/658,248

(22) Filed: May 8, 2024

Related U.S. Application Data
(60) Provisional application No. 63/464,802, filed on May 8, 2023.

(51) Int. Cl. A61B 17/12; A61B 17/00; A61B 90/00; A61L 31/04; A61L 31/08; A61L 31/18; A61M 25/09

(52) U.S. Cl. CPC A61B 17/12159; A61B 17/12036; A61B 17/1204; A61B 17/12109; A61L 31/041; A61L 31/08; A61L 31/18; A61B 2017/00004; A61B 2017/1205; A61B 2090/3966; A61M 2025/09183

(57) ABSTRACT

A system and method for vasculature closure from a single access point are provided, utilizing specifically designed bioresorbable devices. The bioresorbable devices may include a disk-shaped substrate layer with an opening extending from a first surface to a second surface, configured to allow a guidewire to pass through, and a coating configured to encourage adhesion to an interior vessel wall. The system utilizes a guidewire having an expandable portion passing through the opening. The bioresorbable device is carried into the blood vessel within a first tubular member in a compressed configuration, and then ejected from the first tubular member by a second tubular member. The bioresorbable member then assumes a partially expand configuration. The expandable portion of the guidewire can then be used to press the bioresorbable device against an interior vessel wall, allowing it to adhere, after which the expandable portion can be deflated.

### CROSS-REFERENCE TO RELATED APPLICATIONS

The present application claims priority to U.S. Provisional Patent Application No. 63/464,802, filed May 8, 2023, the contents of which are incorporated by reference herein in its entirety.

### TECHNICAL FIELD

The present disclosure is drawn to devices, systems, and method for vasculature closure from a single access point.

### BACKGROUND

Many treatment options require medical instruments to be inserted into a patient's vasculature. The techniques require that some form of vasculature closure in order to achieve hemostasis. There are several categories of conventional vascular access closure devices, including suture-mediated closure devices, mechanical non-suture closure devices, intra-vascular sealant devices, extra-vascular sealant devices and manual compression devices. One technique utilizes a resorbable disc that can be used at the access point, but to use it effectively generally requires another medical instrument be inserted contralaterally in order to provide the necessary force to adhere the disc to a desired location.

What is needed is a device, system, and method that allows for vasculature closure using a single access point.

### BRIEF SUMMARY

A bioresorbable device may be provided to improve vasculature closure. The bioresorbable device may include a disk-shaped substrate layer having fibers. The disk-shaped substrate layer may include a first surface and an opposing second surface. The substrate layer may define an opening extending from the first surface to the second surface, the opening configured to allow a guidewire (such as a 0.035-inch guidewire) to pass through the opening. The opening may be, e.g., a slit having a length of, e.g., 0.5 mm-1 mm. In some embodiments. the substrate forming the substrate layer may be a woven or non-woven substrate. The substrate layer may have a thickness of, e.g., 0.1 mm-0.2 mm. The substrate layer may have an outer diameter of, e.g., 15 mm-25 mm. The substrate layer may include polydioxanone, polycaprolactone, polyglycolide, poly L-lactide, or a combination thereof. In some embodiments, the second surface may be smoother than the first surface.

The bioresorbable device may include a coating on the first surface configured to encourage adhesion to an interior vessel wall. The coating may include collagen, chitin, chitosan, a oligo (ethylene glycol) methylacrylate (OEGMA), methacrylic acid (MAA) or a combination thereof.

The bioresorbable device may include a flap hingedly attached to the first surface or the second surface. The flap may be configured to allow passage of the 0.035-inch guidewire, but close upon removal of the 0.035-inch guidewire. The flap may be adhered onto the substrate layer. The flap may be thermally bound to the substrate layer. The flap may include polydioxanone, polycaprolactone, polyglycolide, poly L-lactide, or a combination thereof.

The bioresorbable device may include a radiopaque material.

In some embodiments, a kit may be provided. The kit may include at least one bioresorbable device of an embodiment as disclosed herein. The kit may include a first tubular member having a proximal end and a distal end, and a first lumen extending therethrough. The kit may include a second tubular member having a proximal end and a distal end, and a second lumen extending therethrough, where the second tubular member may be configured to be slidably positioned within the first lumen. The kit may include a guidewire comprising an expandable portion, where the guidewire may be configured to be slidably positioned within the second lumen. The kit may include an inflation device, which may be configured to be removably couplable to the guidewire.

In some embodiments, the first tubular member may have an outer diameter of 2 mm-2.3 mm. In some embodiments, the first tubular member may include nitinol or a thermoplastic polyurethane (TPU). In some embodiments, the second tubular member may have an outer diameter of 1.4 mm-2 mm. In some embodiments, the second tubular member may include a thermoplastic polyurethane (TPU), a silicone, or a nylon.

In some embodiments, a system may be provided. The system may include a first tubular member having a proximal end and a distal end, and a first lumen extending therethrough. The system may include a second tubular member having a proximal end and a distal end, and a second lumen extending therethrough. The second tubular member may be slidably positioned within the first lumen such that the distal end of the second tubular member is within the first lumen. The system may include an embodiment of a bioresorbable device as disclosed herein positioned within the first lumen, distally from the distal end of the second tubular member. The bioresorbable device may be in a compressed configuration and may be arranged to allow a guidewire to pass through the opening in the bioresorbable device. In some embodiments, the system may include a guidewire having an expandable portion. The guidewire may be slidably positioned within the second lumen. The guidewire may extend through the opening in the bioresorbable device.

In some embodiments, the first tubular member may have an outer diameter of 2 mm-2.3 mm. In some embodiments, the first tubular member may include nitinol or a thermoplastic polyurethane (TPU). In some embodiments, the second tubular member may have an outer diameter of 1.4 mm-2 mm. In some embodiments, the second tubular member may include a thermoplastic polyurethane (TPU), a silicone, or a nylon.

In some embodiments, a method may be provided. The method may include inserting a guidewire comprising an expandable portion into a subject, through an entry point in a blood vessel. The method may include inserting a delivery system over the guidewire, through the entry point, and to a desired location in the blood vessel. The delivery system may include a first tubular member having a proximal end and a distal end, and a first lumen extending therethrough, a second tubular member having a proximal end and a distal end, and a second lumen extending therethrough, where the second tubular member may be slidably positioned within the first lumen such that the distal end may be within the first lumen, and an embodiments of a bioresorbable device as disclosed herein within the first lumen distally from the distal end of the second tubular member, the bioresorbable device being in a compressed configuration, and where the guidewire extends through the opening in the bioresorbable device and through the second lumen. The method may include expanding the expandable portion of the guidewire to a diameter less than a diameter of the blood vessel. The method may include repositioning the second tubular member to cause the distal end of the second tubular member to move distally towards the distal end of the first tubular member, causing the bioresorbable device to exit the first lumen and exhibit a partially expanded configuration. The method may include retracting the guidewire with the expandable portion in a partially expanded position to move the expandable portion into contact with the bioresorbable device, and the bioresorbable device in contact with the first tubular member and/or second tubular member. The method may include retracting the first tubular member, second tubular member, bioresorbable device, and guidewire together until the bioresorbable device is positioned over the entry point. The method may include expanding the expandable portion of the guidewire to fully oppose the bioresorbable device to a wall of the blood vessel. The method may include deflating the expandable portion.

In some embodiments, the method may include inserting an access device prior to inserting the guidewire, and removing the access device after inserting the guidewire. In some embodiments, the method may include removing the first tubular member and second tubular member after the bioresorbable device is positioned over the entry point, leaving the guidewire in place. In some embodiments, the method may include verifying a desired level of hemostasis has been achieved. In some embodiments, the method may include removing the guidewire. In some embodiments, the method may include re-expanding the expandable portion of the guidewire to fully oppose the bioresorbable device to a wall of the blood vessel. In some embodiments, the method may include moving the expandable portion distally from the bioresorbable device and re-expanding the expandable portion of the guidewire.

The opening may be, e.g., a slit having a length of, e.g., 0.5 mm-1 mm. In some embodiments, the substrate forming the substrate layer may be a woven or non-woven substrate. The substrate layer may have a thickness of, e.g., 0.1 mm-0.2 mm. The substrate layer may have an outer diameter of, e.g., 15 mm-25 mm. The substrate layer may include polydioxanone, polycaprolactone, polyglycolide, poly L-lactide, or a combination thereof. In some embodiments, the second surface may be smoother than the first surface.

The bioresorbable device may include a coating on the first surface configured to encourage adhesion to an interior vessel wall. The coating may include collagen, chitin, chitosan, a oligo (ethylene glycol) methylacrylate (OEGMA), methacrylic acid (MAA) or a combination thereof.

The bioresorbable device may include a flap hingedly attached to the first surface or the second surface. The flap may be configured to allow passage of the 0.035-inch guidewire, but close upon removal of the 0.035-inch guidewire. The flap may be adhered onto the substrate layer. The flap may be thermally bound to the substrate layer. The flap may include polydioxanone, polycaprolactone, polyglycolide, poly L-lactide, or a combination thereof.

The bioresorbable device may include a radiopaque material.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a bioresorbable device.

FIG. 2 is an illustration of another embodiment of a bioresorbable device.

FIG. 3 is an illustration of a kit.

FIGS. 4A-4H are cross-sectional views of a portion of a system in use.

FIG. 5 is a flowchart of a method.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

Disclosed is a system and method for vasculature closure, utilizing specifically designed bioresorbable devices, that can be used for closing blood vessels from a single access point.

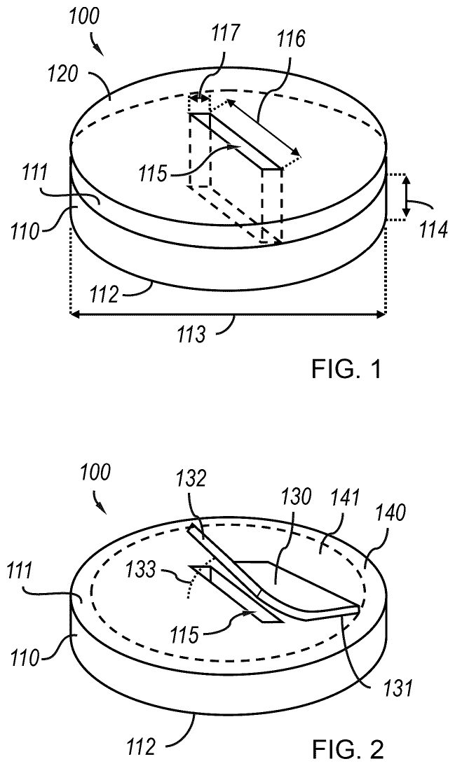

Referring to FIG. 1, a bioresorbable device can be seen. The bioresorbable device (100) may include a substrate layer (110) (such as a disk-shaped substrate layer) comprising fibers. The substrate layer (110) may have a first surface (111) and a second surface (112), the second surface opposite the first surface. In some embodiments, the second surface may be smoother than the first surface. This may be quantified using commercially available measurement tools following standard techniques.

In some embodiments, the substrate layer (110) may have a thickness (114) (e.g., the maximum distance the first surface (111) and second surface (112) may be separated) that may be at least 0.05 mm. In some embodiments, the thickness (114) may be less than 0.5 mm. In some embodiments, the thickness (114) may be from 0.025 mm, 0.05 mm, 0.075 mm, or 0.1 mm up to 0.15 mm, 0.2 mm, 0.3 mm, 0.4 mm, or 0.5 mm, including all combinations and subranges thereof. In some embodiments, the thickness (114) may be 0.1 mm-0.2 mm.

In some embodiments, the outer diameter (113) of the disk-shaped substrate layer (110) may be from 10 mm, 11 mm, 12 mm, 13 mm, 14 mm, or 15 mm, up to 20 mm, 22 mm, 24 mm, 25 mm, or 30 mm, including all combinations and subranges thereof. In some embodiments, the substrate layer may have an outer diameter (113) of 15 mm-25 mm.

In some embodiments, the substrate may be a woven substrate. In some embodiments, the substrate may be comprised of a first plurality of fibers that are substantially oriented in a first direction, and a second plurality of fibers that are substantially oriented in a second direction, the second direction being different from the first direction, where each fiber of the first plurality of fibers may be interwoven with, e.g., three or more fibers from the second plurality of fibers. In some embodiments, the substrate may be a nonwoven substrate. In some embodiments, the fibers may air laid fibers. In some embodiments, the fibers may be spunbond or meltblown fibers.

In some embodiments, the substrate layer (110) may include polydioxanone, polycaprolactone, polyglycolide, poly L-lactide, or a combination thereof. In some embodiments, each fiber may include polydioxanone, polycaprolactone, polyglycolide, poly L-lactide, or a combination thereof.

The disk-shaped substrate layer may define an opening (115) extending from the first surface (111) to the second surface (112). The opening (115) may be configured to allow a guidewire to pass through the opening. In some embodiments, the opening (115) may be configured to allow a 0.035-inch guidewire to pass through the opening.

In some embodiments, the opening (115) may be a slit having a length (116) in a direction parallel to the first surface (111) that is at least 0.1 mm, 0.2 mm, 0.3 mm, 0.4 mm, or 0.5 mm up to 1 mm, 2 mm, 3 mm, 4 mm, or 5 mm, including all combinations and subranges thereof. In some embodiments, the length (116) may be 0.5 mm-1 mm. In some embodiments, the width (117) of the opening (115) may be less than 10% of the length (116). In some embodiments, the width (117) is less than 0.1 mm.

In some embodiments, the opening (115) may be a rectangular slit. In some embodiments, the opening may be circular. In some embodiments, the opening may have an arbitrary shape.

The substrate may include a coating (120) on the first surface (111). As the bioresorbable device may be used in a blood vessel (which may sometimes be referred to herein as a “vessel”), the coating may be configured to encourage adhesion to an interior vessel wall. In some embodiments, the coating may be configured to adhere to an interior vessel wall within short period of time. In some embodiments, that period of time may be less than 2 minutes, less than 1 minute, or less than 30 seconds.

In some embodiments, the coating may include collagen, chitin, chitosan, a oligo (ethylene glycol) methylacrylate (OEGMA), methacrylic acid (MAA), or a combination thereof.

Referring to FIG. 2, the bioresorbable device (100) may include a flap (130) that is hingedly attached to the first surface (111) or the second surface (112) of the substrate layer. In FIG. 2, the flap is shown as having a portion that is attached to the first surface (111) at or near a first end (131) of the flap (130). The flap may be adhered to substrate layer. The flap may be thermally bound to the substrate layer. In some embodiments, the flap may be ultrasonically welded to the substrate layer.

A second end (132) of the flap (130) is shown as being able to be raised 133 off the first surface (111) in order to allow passage of a guidewire, such as a 0.035-inch guidewire, but close when the guidewire is removed.

In some embodiments, the flap may include polydioxanone, polycaprolactone, polyglycolide, poly L-lactide, or a combination thereof. In some embodiments, the flap is composed of an identical material to the substrate layer. In some embodiments, the flap includes at least one material that is not present in the substrate layer.

In some embodiments, the bioresorbable device may include a radiopaque material. In some embodiments, the radiopaque material may be present in a pattern. In some embodiments, the radiopaque material may be present in at least one first portion (140) of the bioresorbable device, while at least one second portion (141) is free of radiopaque material. In some embodiments, the radiopaque material may be present in at least a portion of the flap (130). In some embodiments, the radiopaque material may define a ring around the opening (115).

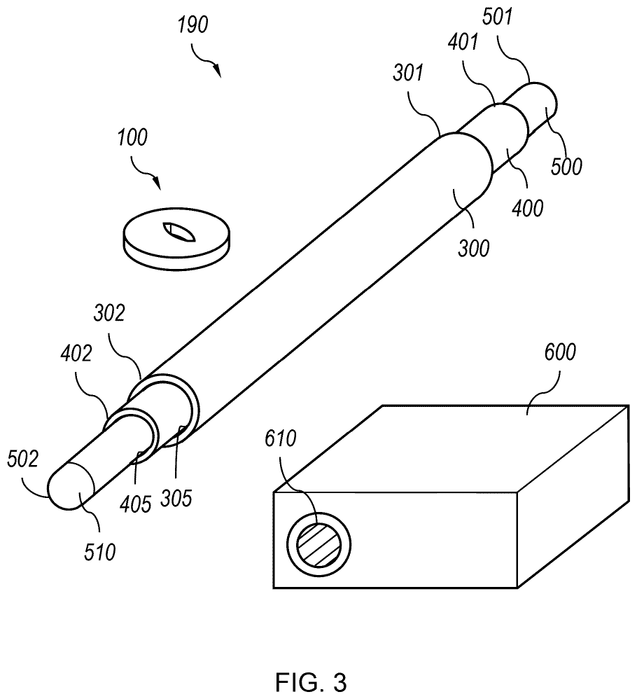

Referring to FIG. 3, additional components, such as those forming a kit, may be provided. In some embodiments, a kit (190) may include an embodiment of a bioresorbable device (100) as disclosed herein.

The kit may include a first tubular member (300) having a proximal end (301) and a distal end (302), and a first lumen (305) extending therethrough.

In some embodiments, the first tubular member may have an outer diameter that is from 1.5 mm, 1.6 mm, 1.7 mm, 1.8 mm, 1.9 mm, or 2 mm up to 2.1 mm, 2.2 mm, 2.3 mm, 2.4 mm, or 2.5 mm, including all combinations and subranges thereof. In some embodiments, the first tubular member may have an outer diameter of 2 mm-2.3 mm.

In some embodiments, the first tubular member may include nitinol, or a thermoplastic polyurethane (TPU).

The kit may include a second tubular member (400) having a proximal end (401) and a distal end (402), and a second lumen (405) extending therethrough, the second tubular member (400) configured to be slidably positioned within the first lumen (305).

In some embodiments, the second tubular member may have an outer diameter that is from 1 mm, 1.1 mm, 1.2 mm, 1.3 mm, 1.4 mm, or 1.5 mm up to 1.6 mm, 1.7 mm, 1.8 mm, 1.9 mm, 2 mm, 2.1 mm, or 2.2 mm, including all combinations and subranges thereof. In some embodiments, the second tubular member may have an outer diameter of 1.4 mm-2 mm.

In some embodiments, the second tubular member may include a thermoplastic polyurethane (TPU), a silicone, or a nylon.

The kit may include a guidewire (500) having a proximal end 501 and a distal end 502, the guidewire (500) configured to be slidably positioned within the second lumen (405). The guidewire may include an expandable portion (510). In some embodiments, the expandable portion (510) is positioned at or near the distal end 502 of the guidewire (500).

The kit may include an inflation device 600. The inflation device 600 may be configured to be removably couplable to the guidewire (500). In some embodiments, the inflation device may contain one or more ports 610 that are configured to be operably coupled to the guidewire (500). In some embodiments, the inflation device may be configured to provide a predefined quantity of a fluid to the expandable portion (510) of the guidewire (500).

The inflation device may be configured to inflate and deflate the expandable portion of the guidewire.

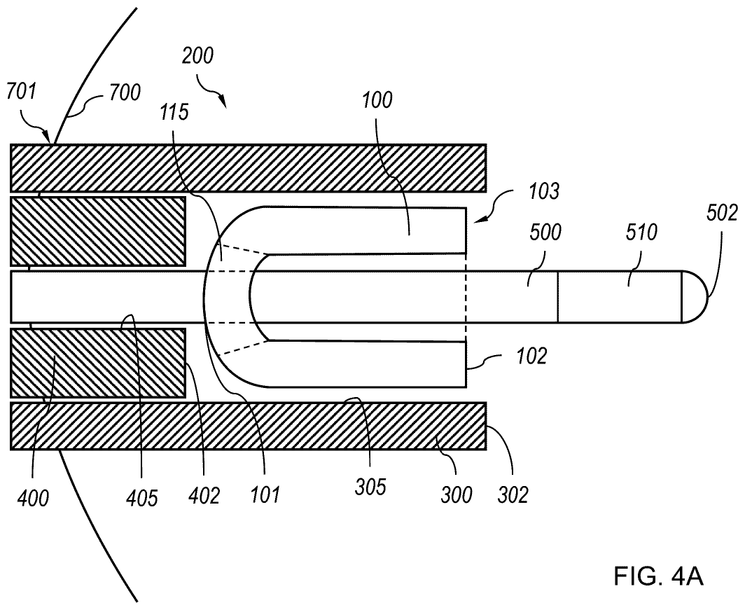

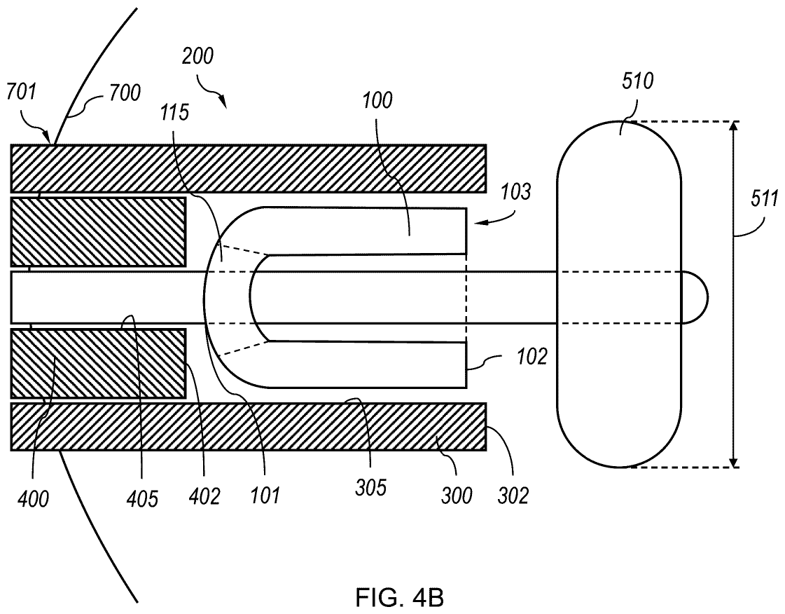

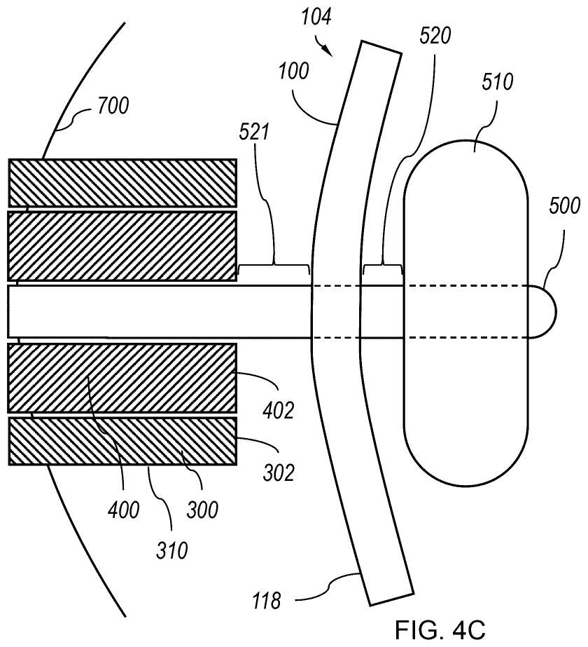

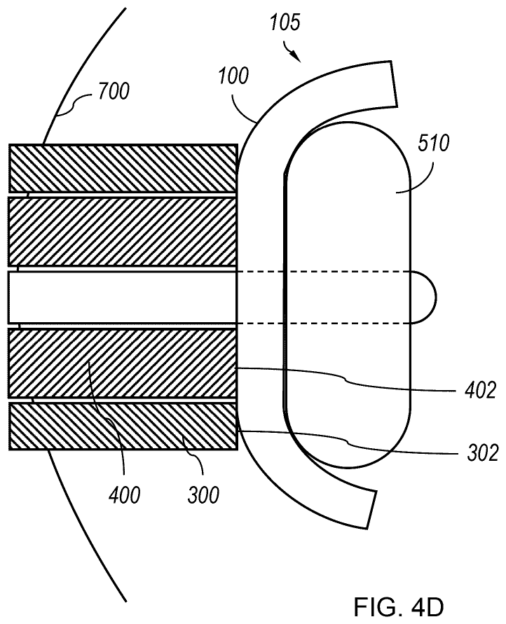

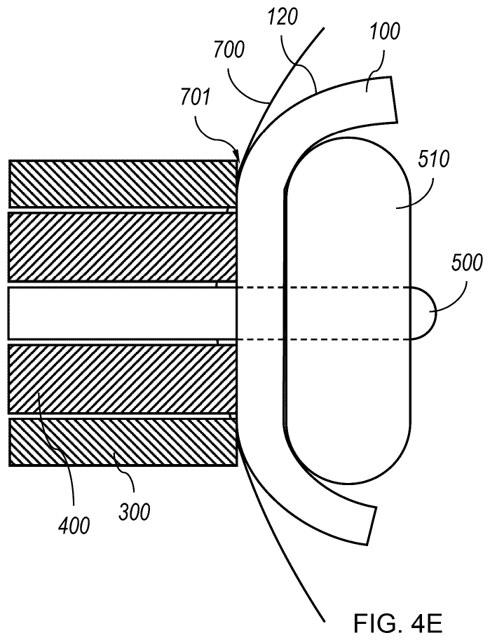

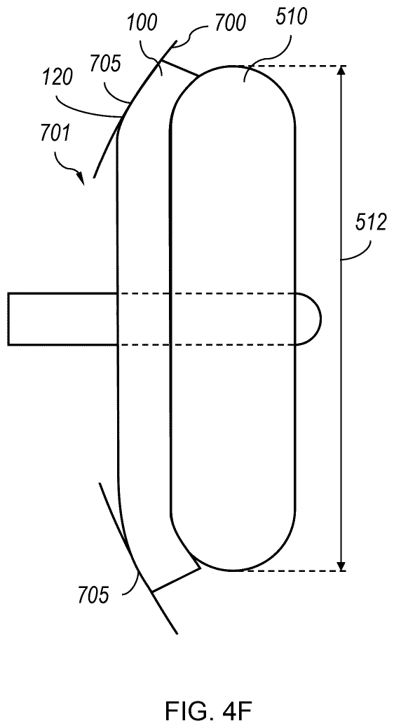

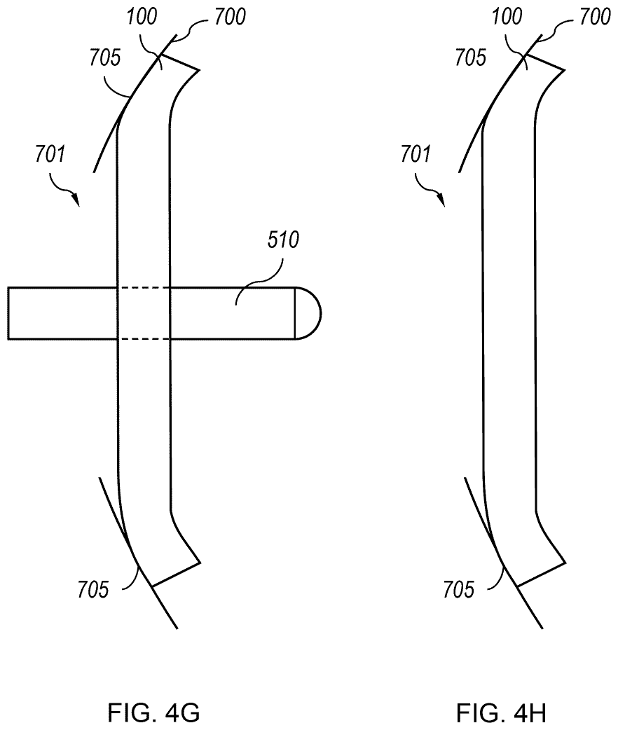

In some embodiments, a system may be provided. Referring to FIGS. 3 and 4A, in some embodiments, a system (200) may include a first tubular member (300) having a proximal end (301) and a distal end (302), and a first lumen (305) extending therethrough.

In some embodiments, the first tubular member may have an outer diameter that is from 1.5 mm, 1.6 mm, 1.7 mm, 1.8 mm, 1.9 mm, or 2 mm up to 2.1 mm, 2.2 mm, 2.3 mm, 2.4 mm, or 2.5 mm, including all combinations and subranges thereof. In some embodiments, the first tubular member may have an outer diameter of 2 mm-2.3 mm.

In some embodiments, the first tubular member may include nitinol, or a thermoplastic polyurethane (TPU).

The system may include a second tubular member (400) having a proximal end (401) and a distal end (402). In some embodiments, the second tubular member (400) may be slidably positioned within the first lumen (305) such that the distal end (402) is within the first lumen (305). In some embodiments, the distal end (402) of the second tubular member (400) may be positioned proximally from the distal end (302) of the first tubular member (300).

In some embodiments, the second tubular member may have an outer diameter that is from 1 mm, 1.1 mm, 1.2 mm, 1.3 mm, 1.4 mm, or 1.5 mm up to 1.6 mm, 1.7 mm, 1.8 mm, 1.9 mm, 2 mm, 2.1 mm, or 2.2 mm, including all combinations and subranges thereof. In some embodiments, the second tubular member may have an outer diameter of 1.4 mm-2 mm.

In some embodiments, the second tubular member may include a thermoplastic polyurethane (TPU), a silicone, or a nylon.

The system may include an embodiment of a bioresorbable device (100) as disclosed here. The bioresorbable device may be positioned within the first lumen (305). In some embodiments, the bioresorbable device may be positioned such that the distal end (102) of the bioresorbable device in the first lumen is proximal from the distal end (302) of the first tubular member (300). The bioresorbable device may be positioned distally from the distal end (402) of the second tubular member (400). In some embodiments, the proximal end (101) of the bioresorbable device in the first lumen may be in contact with the distal end (402) of the second tubular member (400). In some embodiments, the proximal end (101) of the bioresorbable device in the first lumen may be positioned distal from the distal end (402) of the second tubular member (400).

The bioresorbable device may be in a first configuration (103). The first configuration may be a compressed configuration. The compressed configuration may be configured such that the opening (115) is positioned at or near the proximal end (101) of the bioresorbable device (100). The compressed configuration may be configured such that the opening (115) is positioned at or near the distal end (102) of the bioresorbable device (100).

The bioresorbable device may be configured to allow a guidewire (500) to pass through the opening (115) in the bioresorbable device (100).

The system may include a guidewire (500). The guidewire may include an expandable portion (510). The guidewire may be slidably positioned within the second lumen (405). The guidewire may extend through the opening (115) in the bioresorbable device (100). The guidewire may be positioned such that the expandable portion (510) is positioned distal from the distal end (102) of the bioresorbable device (100) in the first lumen.

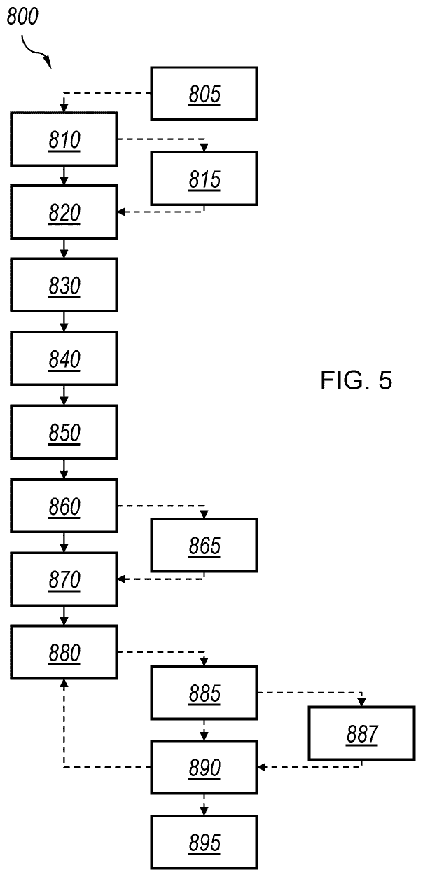

Referring to FIG. 5, and also to FIGS. 4A-4F, a method may be provided for vasculature closure.

The method (800) may include inserting (810) a guidewire (500) that may include an expandable portion (510) into a subject, through an entry point (701) in a blood vessel (700).

The method may include inserting (820) a delivery system over the guidewire, through the entry point (701), and to a desired location in the blood vessel (700). The delivery system may include an embodiment of a system as disclosed herein. In some embodiments, the delivery system includes a first tubular member (300) having a proximal end and a distal end, and a first lumen extending therethrough, a second tubular member (400) having a proximal end and a distal end, and a second lumen extending therethrough, and an embodiment of a bioresorbable device as disclosed herein, positioned within the first lumen distally from the distal end of the second tubular member, the bioresorbable device being in a compressed configuration. The second tubular member may be slidably positioned within the first lumen such that the distal end is within the first lumen. The guidewire may extend through the opening in the bioresorbable device and through the second lumen. At this step, as seen in FIG. 4A, the bioresorbable device (100) is a first (e.g., compressed) configuration (103) within the first lumen, inside blood vessel (700). The expandable portion is in a non-expanded (e.g., compressed) configuration.

The method may include expanding (830) the expandable portion (510) of the guidewire to a diameter (511) that is less than a diameter of the blood vessel. In some embodiments, this may be a partially expanded diameter. At this step, as seen in FIG. 4B, the bioresorbable device (100) is a first (e.g., compressed) configuration (103) within the first lumen, inside blood vessel (700), while the expandable portion is in a partially expanded configuration.

The method may include repositioning (840) the second tubular member (400) to cause the distal end (402) of the second tubular member (400) to move distally towards the distal end (302) of the first tubular member (300), causing the bioresorbable device to exit the first lumen and exhibit a second configuration (104). The second configuration may be a partially expanded configuration. At this step, as seen in FIG. 4C, the bioresorbable device (100) has been moved distal from the distal end of the first lumen, and is now in a second (e.g., partially expanded) configuration (104), and the expandable portion is in a partially expanded configuration.

In some embodiments, the partially expanded configuration (104) is configured such that at least a portion (118) of the substrate layer extends radially beyond the outer surface (310) of the first tubular member (300). In some embodiments, the expandable portion may be separated from the bioresorbable device (100) by a first distance (520). In some embodiments, the distance may be at least 1 mm. In some embodiments, the expandable portion may be separated from the distal end (302) of the first tubular member by a second distance (521). In some embodiments, the distance may be at least 1 mm. In some embodiments, the first distance is greater than the second distance. In some embodiments, the first distance is less than the second distance. In some embodiments, the first distance is substantially equal to the second distance (e.g., the distances are within 5% of each other).

The method may include retracting 850 the guidewire with the expandable portion in a partially expanded position to move the expandable portion into contact with the bioresorbable device, and the bioresorbable device in contact with the first tubular member and/or second tubular member. This may be seen in FIG. 4D—the partially expanded expandable portion (510) may be in contact with the bioresorbable device (100). The bioresorbable device may be in a third configuration (105), different from the second configuration (104).

The method may include retracting (860) the first tubular member (300), second tubular member (400), bioresorbable device (100), and guidewire (500) together until the bioresorbable device is positioned over the entry point. This is seen in FIG. 4E, where the system is retracted until the bioresorbable device (100) is positioned over the entry point (701). In some embodiments, the coating is facing the blood vessel (700). In some embodiments, a portion of the coating is in contact with the blood vessel.

The method may include expanding (870) the expandable portion of the guidewire to fully oppose the bioresorbable device to a wall of the blood vessel. This is seen in FIG. 4F, where the diameter (512) of the expandable portion is increased, and at least a portion (705) of the blood vessel wall around the entry point (701) is in contact with the coating (120) on the bioresorbable device.

The method may include deflating (880) the expandable portion. This can be seen in FIGS. 4G-4H. The bioresorbable device remains in place over the entry point after the partially expandable portion (510) has been deflated.

In some embodiments, the method may include removing (895) the guidewire.

In some embodiments, the method may include inserting (805) an access device into the patient, and optionally into the entry point, prior to inserting the guidewire. In some embodiments, the method may include removing (815) the access device after inserting the guidewire. In FIG. 5, this is shown as occurring immediately after insertion of the guidewire, but this could occur at any later step in the process up to the point at which the guidewire is removed (see verifying (885) step).

In some embodiments, the method may include removing (865) the first tubular member (300) and second tubular member (400) after the bioresorbable device is positioned over the entry point, leaving the guidewire (500) in place. This can be seen in FIG. 4F, where by the time the expandable portion is fully expanded to a diameter (512), the first and second tubular members have been removed.

In some embodiments, the method may include verifying (885) that a desired level of hemostasis has been achieved.

In some embodiments, the method may include re-expanding (890) the expandable portion of the guidewire to fully oppose the bioresorbable device to a wall of the blood vessel.

In some embodiments, the method may include moving (887) the expandable portion distally from the bioresorbable device and re-expanding (890) the expandable portion of the guidewire. The deflating, verifying, and moving/reinflating steps may be repeated until the desired level of hemostasis is achieved, or until alternative approaches are determined to be necessary.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

1. A bioresorbable device, comprising:
a disk-shaped substrate layer comprising fibers, the disk-shaped substrate layer having a first surface and a second surface opposing the first surface, and the disk-shaped substrate layer having an opening extending from the first surface to the second surface configured to allow a guidewire to pass through the opening; and
a coating on the first surface configured to encourage adhesion to an interior vessel wall.

2. The bioresorbable device of claim 1, wherein the disk-shaped substrate layer is a woven substrate.

3. The bioresorbable device of claim 1, wherein the disk-shaped substrate layer is a nonwoven substrate.

4. The bioresorbable device of claim 1, wherein the opening is configured to allow a 0.035-inch guidewire to pass through the opening.

5. The bioresorbable device of claim 1, wherein the disk-shaped substrate layer has a thickness of 0.1 mm-0.2 mm.

6. The bioresorbable device of claim 1, wherein the disk-shaped substrate layer has a diameter of 15 mm-25 mm.

7. The bioresorbable device of claim 1, wherein the opening is a slit having a length in a direction parallel to the first surface of 0.5 mm-1 mm.

8. The bioresorbable device of claim 1, wherein the disk-shaped substrate layer comprises polydioxanone, polycaprolactone, polyglycolide, poly L-lactide, or a combination thereof.

9. The bioresorbable device of claim 1, wherein the second surface is smoother than the first surface.

10. The bioresorbable device of claim 1, wherein the coating comprises collagen, chitin, chitosan, a oligo (ethylene glycol) methylacrylate (OEGMA), methacrylic acid (MAA) or a combination thereof.

11. The bioresorbable device of claim 1, further comprising a flap that is hingedly attached to the first surface or the second surface, the flap configured to allow passage of a 0.035-inch guidewire, but close upon removal of the 0.035-inch guidewire.

12. The bioresorbable device of claim 11, wherein the flap is adhered onto the disk-shaped substrate layer.

13. The bioresorbable device of claim 11, wherein the flap is thermally bound to the disk-shaped substrate layer.

14. The bioresorbable device of claim 11, wherein the flap comprises polydioxanone, polycaprolactone, polyglycolide, poly L-lactide, or a combination thereof.

15. The bioresorbable device of claim 1, wherein the disk-shaped substrate layer comprises a radiopaque material.

16. A kit, comprising:
at least one bioresorbable device of claim 1;
a first tubular member having a proximal end and a distal end, and a first lumen extending therethrough;
a second tubular member having a proximal end and a distal end, and a second lumen extending therethrough, the second tubular member configured to be slidably positioned within the first lumen;
a guidewire comprising an expandable portion, the guidewire configured to be slidably positioned within the second lumen; and
an inflation device configured to be removably couplable to the guidewire.

17-20. (canceled)

21. A system, comprising:
a first tubular member having a proximal end and a distal end, and a first lumen extending therethrough;
a second tubular member having a proximal end and a distal end, and a second lumen extending therethrough, the second tubular member slidably positioned within the first lumen such that the distal end is within the first lumen; and
a bioresorbable device of claim 1 positioned within the first lumen distally from the distal end of the second tubular member, the bioresorbable device being in a compressed configuration and arranged to allow a guidewire to pass through the opening in the bioresorbable device.

22-25. (canceled)

26. The system of claim 21, further comprising a guidewire comprising an expandable portion, the guidewire slidably positioned within the second lumen and extending through the opening in the bioresorbable device.

27. A method, comprising:
inserting a guidewire comprising an expandable portion into a subject, through an entry point in a blood vessel;
inserting a delivery system over the guidewire, through the entry point, and to a desired location in the blood vessel, the delivery system comprising:
a first tubular member having a proximal end and a distal end, and a first lumen extending therethrough;
a second tubular member having a proximal end and a distal end, and a second lumen extending therethrough, the second tubular member slidably positioned within the first lumen such that the distal end is within the first lumen; and
a bioresorbable device of claim 1 positioned within the first lumen distally from the distal end of the second tubular member, the bioresorbable device being in a compressed configuration, where the guidewire extends through the opening in the bioresorbable device and through the second lumen;
expanding the expandable portion of the guidewire to a diameter less than a diameter of the blood vessel;
repositioning the second tubular member to cause the distal end of the second tubular member to move distally towards the distal end of the first tubular member, causing the bioresorbable device to exit the first lumen and exhibit a partially expanded configuration;
retracting the guidewire with the expandable portion in a partially expanded position to move the expandable portion into contact with the bioresorbable device, and the bioresorbable device in contact with the first tubular member and/or second tubular member;
retracting the first tubular member, second tubular member, bioresorbable device, and guidewire together until the bioresorbable device is positioned over the entry point;
expanding the expandable portion of the guidewire to fully oppose the bioresorbable device to a wall of the blood vessel; and
deflating the expandable portion.

28-44. (canceled)
